(19) United States
(12) Patent Application Publication
(10) Pub. No.: US 2010/0305305 A1
(43) Pub. Date: Dec. 2, 2010

(54) METHOD FOR PURIFYING FACTOR VIII AND VON WILLEBRAND FACTOR

(75) Inventors: Michel Poulle, Wavrin (FR); Patrick Bonneel, Lille (FR)

Correspondence Address: YOUNG & THOMPSON, 209 Madison Street, Suite 500, Alexandria, VA, 22314, US

(73) Assignee: LFB-BIOTECHNOLOGIES, Les Ulis (FR)

(21) Appl. No.: 12/675,608

(22) PCT Filed: Aug. 28, 2008
(86) PCT No.: PCT/FR08/51543

§ 371 Date: Feb. 26, 2010

(30) Foreign Application Priority Data
Aug. 30, 2007 (FR) 0757266

(51) Int. Cl. C07K 14/755; C07K 14/745

(52) U.S. Cl. 530/383

(57) ABSTRACT

The purification method includes, starting from a solution selected from (i) a solution containing a mixture of FVIII and FvW, (ii) a solution containing FvW, (iii) a solution derived from a secretion of a non-human animal and (iv) a solution derived from a FVIII-containing plant extract, a step of absorption of FVIII or FvW on an ion-exchange chromatography filter-type membrane.

### FIELD OF THE INVENTION

The field of the present invention relates to the purification of factor VIII and of Von Willebrand factor, to be used as active agents for a drug.

### PRIOR ART

Factor VIII (hereunder also referred to as “FVIII”) is a plasma protein present in the human plasma in a low concentration. However, it represents the key point in the clotting cascade. Indeed, this protein acts as a cofactor of factor IX (or “FIX”) so as to activate factor X (or “FX”). Once activated, factor X converts prothrombin to thrombin, which in turn converts fibrinogen to fibrin, thus leading to the haemostatic fibrin clot formation.

People suffering from haemophilia A have a FVIII deficiency, that causes severe bleeding, either spontaneously, or following a trauma of accidental or surgical origin.

Those individuals are traditionally treated by injecting purified plasma-derived FVIII. Since such injections are often numerous and repeated, it is crucial to have highly pure FVIII concentrates available. Indeed, if the FVIII concentrates are insufficiently purified, they may contain high amounts of fibrinogen and immunoglobulins, that are likely to induce undesirable immune responses.

The provision of plasma-derived proteins to be used for therapeutic purposes thus requires plasma-derived FVIII purification methods to obtain high-purity products.

Factor VIII concentrates are most often prepared from a cryoprecipitated human plasma fraction. The purity of the Factor VIII concentrates that are generally obtained in the industrial centers for treating human plasma is often of about 1 IU/mg and does not generally excess the maximum range of from 10 to 20 IU/mg. The most common production methods do imply precipitation steps which aim at removing, often very insufficiently, the protein-derived contaminants such as fibrinogen, fibronectin, and immunoglobulins. These methods may use or combine a low-temperature precipitation (10° C.), or the addition of protein-precipitating agents, like hydrophilic polymers such as PEG (Newman and al., Br. J. Haematol 21:1-20, 1971; Hao and al., in Methods of Plasma Protein Fractionation, Academic Press 1980, pp. 57-74), polyvinylpyrrolidone (Casillas and Simonetti, Br. J. Haemato, 50:665-672, 1982), dextran, Ficoll, Percoll, hydroxyethyl starch and alumina were suggested as precipitation agents. The same does apply as regards the use of glycine and sodium chloride recommended by Thorell and Blombäck (Thorell and Blombäck, Thromb Res. 1984 Aug. 15; 35(4):431-50). Similarly, some authors (Ng and al, Thrombosis Res., 42:825-834, 1986) did succeed in combining three precipitation agents that are PEG, glycine, and sodium chloride to obtain Factor VIII concentrates with a specific activity ranging from 10 to 16 IU/mg.

Factor VIII concentrates were also produced by including to the production procedure a contact step with porous silica beads intended to entrap the protein-derived contaminants with a low molecular weight (Margolis and al., Vox Sang. 46:341-348, 1984). The specific activity of the product still remains relatively poor: 1 UI/mg.

Methods for preparing Factor VIII concentrates with very high purity appeared. Indeed, concentrates obtained by means of immunoaffinity chromatography methods were proposed (Zimmerman and Fulcher, Thrombosis Res., Suppl. VII, p. 58, 1987; Berntorp and Nilsson, Thrombosis Res., Suppl. VII, p. 60, 1987; Levine and al., Thombosis Res, Suppl. VII, 1987). Such methods do consist in purifying factor VIII by means of anti-Factor VIII:C or anti-Von Willebrand factor antibodies that were fixed on a chromatographic substrate. Such methods are efficient but do require the use of drastic solutions to desorb factor VIII either from its antibody or from Von Willebrand factor. An additional ultrafiltration step aiming at removing the undesirable chemical agents is therefore necessary but it may affect the biological activity of factor VIII. The specific activity of factor VIII may reach 4000 to 10000 IU/mg during production, but its instability requires a stabilizing agent to be added, such as albumin, prior to the freeze-drying step, which reduces the specific activity of factor VIII to 3-5 IU/mg. However, the major drawback of the immunoaffinity purification lies in the presence of residual antibodies that are of the animal origin and thus may cause in the patients the incidence of immune reactions against those non-human proteins.

Therefore, all these methods do not allow to provide Factor VIII concentrates with a very high degree of purity, that are fully free of non-human proteins such as animal-derived antibodies, by means of methods applicable to a large-scale industrial environment.

EP 0 343 275 describes a method for preparing Factor VIII from a cryoprecipitate characterized in that, prior to the viral inactivation treatment, the cryoprecipitate is suspended in water containing from 1 to 3 U/ml of heparin at a pH value ranging from 6.5 to 7.5, is reacted with an aluminium hydroxide suspension and after cooling at a temperature of from 10 to 18° C. and adjustment of the pH to a value ranging from 6 to 7, is centrifuged or filtered, then the purification is further conducted with a post-treatment, in particular by means of a chromatography using an ion-exchange resin such as Fractogel-DEAE (now called DEAE-TOYOPEARL®, marketed by the Tosoh Bioscience company), of the hydrophilic type.

This document thus describes the purification of the only Factor VIII by associating a very particular preliminary step, especially characterized by the total lack of any ethanol treatment, with an ion-exchange chromatography on a hydrophilic-type resin such as Fractogel DEAE.

EP 0 359 593 describes an anion-exchange chromatography purification method, which enables to separate on a single chromatographic column the expected proteins under conditions that are sufficiently well pondered to make any post-treatment unnecessary. Such method makes it possible to separate Factor VIII, fibrinogen, fibronectin and Von Willebrand factor proteins from human or animal plasma. Such method may be summarized as follows: the cryoprecipitate fraction that has been solubilized in water is subjected to a single separation by means of a chromatography using an anion-exchange resin which matrix is of the macro-crosslinked vinyl polymer gel type, capable, due to its porosity properties, to retain the Factor VIII-Von Willebrand factor complex, then the various proteins are selectively collected by successive increases of the ionic strength of the elution buffer. Such method gives good results by using DEAE moieties grafted onto a Fractogel® TSK-DEAE 650 resin (now called DEAE-TOYOPEARL®, marketed by the Tosoh Bioscience company). However, such method, while enabling to obtain purified FVIII, does not enable to obtain sufficiently purified Von Willebrand factor.

Otherwise Von Willebrand factor (hereunder also referred to as “FvW”) does play a crucial role in haemostasis by exerting two distinct functions: as an adhesion protein, it allows blood platelets to disperse, to adhere and to aggregate onto the vascular subendothelium, and thus takes part to the fast cicatrization of injured vessels and, on the other hand, it does ensure the stabilization and the transportation of factor VIII into the blood circulation, to which it is non covalently associated.

A FvW congenital deficiency or a structural defect of this factor does cause von Willebrand disease which does express as skin and mucosal bleedings. The clinical expression of this disease is very heterogeneous and is very problematic when a surgery has to take place. The treatment of von Willebrand disease is imperative to correct primary haemostasis (bleeding time) and clotting defects.

Treatment of the disease is traditionally effected as a substitution therapy using FvW-enriched human plasma derivatives (for example the cryoprecipitated fraction of the plasma or Factor VIII concentrates containing sufficiently FvW associated to the same).

But FvW is a protein that is difficult to purify. Indeed, Von Willebrand factor is the largest protein known which circulates in plasma. It is composed of a set of disulfide bridge-linked multimers, which base element has a molecular weight of about 260 kilodaltons (kDa). FvW's smallest form, in plasma, is a dimer of from 440 to 500 kDa and the largest forms are multimers of said dimer which molecular weight can reach up to 20 millions Daltons. Such arrangement of the subunits in multimers may be specific for cells in which it has been formed: FvW is synthesized and polymerized in megakaryocytes and in endothelial cells.

Thus, due to its complexity and because of its bond to FVIII, the Von Willebrand factor molecule is very complicated to prepare.

Various methods for preparing FvW concentrates typically combine steps consisting in precipitating a plasma fraction so as to remove the main part of the unwanted proteins (fibrinogen, fibronectin, and the like), and/or chromatographic steps (ion-exchange chromatography, affinity chromatography, immunoaffinity chromatography, steric exclusion chromatography, and the like) aiming at providing highly pure concentrates with a high specific activity, while preserving the integrity of the multimer forms, especially of those of high molecular weight, the biological action of which is decisive on the healing processes.

The European patent EP 0 503 991 discloses an industrial-scale method for preparing a FvW concentrate comprising a pre-purification step of a plasma cryoprecipitated fraction and three successive chromatography steps, the third one being an affinity chromatography performed on an agarose fixed-gelatin column. The thus obtained FvW concentrate has a specific activity higher than 100 VWF: RCo/mg as expressed in activity units of ristocetin cofactor per mg of proteins, and a level of high molecular weight multimers similar to that of the initial plasma.

The European patent application EP 0 934 748 describes a FvW preparation method comprising the combination of anion-exchange and cation-exchange chromatographies. The resulting FvW fractions have a specific activity higher than 100 IU FvW:Ag/mg as expressed in FvW antigen units per mg of protein, but still contain significant amounts of Factor VIII.

The U.S. Pat. No. 6,579,723 describes a method for preparing highly purified FvW by immunoaffinity chromatography where the immunoadsorbents are anti-FvW antibodies. An additional step of purification by affinity chromatography on heparin may also be provided. However, the drawback of such immunoaffinity purification is the possible presence of residual antibodies that may induce immune reactions.

The European patent EP 0 383 234 does teach the preparation of a FvW concentrate by means of an anion-exchange chromatography, performed with acidic solutions (pH ranging from 5.5 to 6.5) containing carbohydrates, so as to fix factor VIII on the anion exchanger. To recover FvW, together with non retained fibronectin and fibrinogen by washing the substrate does require additional precipitation steps to isolate a purified FvW concentrate.

EP 1 632 501 describes a method for preparing a highly pure Von Willebrand concentrate from a Von Willebrand factor-containing biological fraction, comprising the separation by an anion-exchange chromatography using a mild base type-, vinyl polymer substrate. Advantageously, such method can be carried out very easily and makes it possible to obtain a highly specific Von Willebrand factor containing very few factor VIII.

FVIII and FvW are very useful plasma-derived proteins and deficiencies thereof for some people do lead to serious haemostasis disorders. It is therefore utmost important to develop methods for preparing these proteins, capable of producing products with a purity degree adapted to the repeated use for patients.

The methods described in the previous art enable either to obtain pure factor VIII, but poorly pure Von Willebrand factor, or to obtain both pure FVIII and FvW, provided however that a complicated and cumbersome method is implemented.

### SUMMARY OF THE INVENTION

The present invention relates to a method for purifying a solution containing a mixture of FVIII and FvW, or a FvW-containing solution or a solution derived from a secretion of an animal, in particular a non-human animal, or a FVIII-containing plant extract, characterized in that it comprises a chromatography step on an ion-exchange chromatography filter-type membrane that can adsorb at least one protein selected from FVIII and FvW.

### DESCRIPTION OF THE INVENTION

Therefore, it is an object of the present invention to provide a method for purifying FVIII or FvW from a solution selected from (i) a solution containing a mixture of FVIII and FvW, (ii) a solution containing FvW, (iii) a solution derived from a secretion of a non-human animal and (iv) a solution derived from a FVIII-containing plant extract, said method being characterized in that it comprises a step of adsorption of FVIII or FvW on an ion-exchange chromatography filter-type membrane.

As used herein, a “purification method” is intended to mean a method for separating FVIII from the other molecules present in the medium, or a method for separating FvW from the other molecules present in the medium, or a method for separating FVIII from FvW, or a method for separating FVIII/FvW complexes from the other molecules present in the medium. Those molecules may be proteins which differ from FVIII and FvW, viruses, bacteria, spores, culture medium, foetal calf serum, this list being non limitative.

As used herein, “FVIII” is intended to mean any form of FVIII, being especially capable of acting as a cofactor in the FIX activation and capable of forming a complex with FvW, notably with mature FVIII, mature FVIII biologically active derivatives such as pro-FVIII which contains the pro-peptide (pro-FVIII), the protein constructs comprising non-mature FvW, the FVIII precursor (pre-pro-FVIII), mature FVIII obtained after cleavage of the signal peptide and the pro-peptide. Other FVIII biologically active derivatives included in the present invention are pro-drugs undergoing post-translational modifications or which are converted into biologically active forms, such as truncated forms, deleted forms, for example FVIII deleted from one or more aminoacids located in the region between Arg-759 and Ser-1709 described in EP 218 712, chimeric forms, and forms comprising post-translational modifications which differ from plasma natural mature forms. These various FVIII forms may for example be produced by modifying mature FVIII or any other form that does naturally occur in blood. The nucleotide sequence encoding such a FVIII may be derived from various sources, preferably from mammals, including the human, porcine, ovine, bovine, Equidae and Caprinae sources, this list being non limitative.

As used herein, “FvW” is intended to mean any FvW form, especially mature FvW, mature FvW biologically active derivatives, such as pro-FvW which contains the pro-peptide, the protein constructs comprising non mature FvW, including the FvW precursor (pre-pro-FvW), the FvW propeptide (pro-FvW), mature FvW obtained after cleavage of the signal peptide and pro-peptide. Other FvW biologically active derivatives included in the present invention are pro-drugs undergoing post-translational modifications or which are converted to biologically active forms, such as truncated forms, deleted forms, for example the A2 domain-deleted FvW which is proteolysis-resistant (Lankhof and al., Thromb. Haemost. 77: 1008-1013, 1997), the FvW fragment between Val 449 and Asn 730 including the glycoprotein 1b-binding domain and binding sites for collagen and heparin (Pietu and al., Biochem. Biophys. Res. Commun. 164: 1339-1347, 1989), chimeric forms, and forms comprising post-translational modifications which differ from plasma natural mature forms. These various FvW forms may for example be prepared by modifying mature FvW or any other form that does naturally occur in blood. The nucleotide sequence encoding such a FvW may be derived from various sources, preferably from mammals, including the human, porcine, ovine, bovine, Equidae and Caprinae sources, this list being non limitative.

As used herein, a “solution containing a mixture of FVIII and FvW” is intended to mean any solution containing FVIII and FvW, in a complex state or a separate state. These solutions may be of plasmatic or recombinant or transgenic origin.

When the solution is of recombinant origin, it is derived from a unicellular system, wherein the FVIII and FvW protein expression has been induced. It can be mentioned any animal or human cell line that has been transfected with a vector comprising the gene encoding each of these proteins, preferably the gene encoding the human protein, where said cell lines may be especially selected from CHO-K, CHO-LeC10, CHO Lec-1, CHO Pro-5, CHO dhfr-, Wil-2, Jurkat, Vero, Molt-4, COS-7, 293-HEK, YB2/0 (ATCC CRL-1662), BHK, K61-16, NSO, SP2/0-Ag 14 and P3X63Ag8. 653, SK-Hep, HepG2, PERC6 (Crucell) cell lines, as well as plant cells, bacteria systems, for example E. Coli, fungi systems, systems using viruses, especially baculoviruses, this list being non limitative.

Such cellular systems do express FvW and FVIII proteins by means of methods well known from the man skilled in the art. U.S. Pat. No. 5,198,349 may be mentioned as an example, which content is incorporated herein as a reference. Said document describes the FVIII and FvW co-expression, especially in CHO cells co-transfected with, on the one hand, an expression vector into which the coding sequence of FVIII was inserted, and, on the other hand, an expression vector into which the FvW coding sequence was inserted.

When the solution is of transgenic origin, it is derived from a pluricellular system, especially from an animal or a plant obtained by transgenesis, that is to say wherein one or several cell(s) received a recombinant DNA molecule. Suitable examples thereof include dogs, cats, mice, rats, hamsters, cows, goats, sheeps, rabbits and pigs, horses, insects, plants, for example tobacco, soybean, this list being non limitative.

When FVIII and FvW are produced by an animal, this production may occur in various media secreted by the animal, for example urine, blood, saliva or milk, this list being non limitative. Such production methods may be carried out by means of methods well known from the man skilled in the art. EP 0 741 515 and EP 807 170 may be mentioned as examples, this list being non limitative. The latter describes the production of a transgenic animal that did integrate in a stable manner in its genome the DNA molecules encoding FVIII and FvW, so as to express both of them and to secrete them in the milk.

When FVIII and FvW are produced by a plant, this production may be carried out by means of methods well known from the man skilled in the art, as described for example in U.S. Pat. No. 6,331,416 and U.S. Pat. No. 5,994,628, this list being non limitative.

When the solution is of plasmatic origin, it may be either animal- or human-derived plasma, or a cryoprecipitate, or a fraction obtained by standard fractionation methods (Cohn and al., J. Am. Chem. Soc., 68, 459, 1946 and Kistler and al., Vox Sang., 7, 1962, 414-424). These fractions may optionally have been subjected to a pre-purification treatment such as the adsorption on aluminium hydroxide.

The FVIII and FvW mixture means that these proteins may be present in approximately similar amounts, or that a protein is predominantly present, or even very predominantly present as compared to the other.

As used herein, a “solution containing FvW” is intended to mean any solution containing FvW, and substantially devoid of, that is to say with little or even very little FVIII. This solution may be of recombinant, transgenic or plasmatic origin.

When the solution is of recombinant origin, it is derived from an unicellular system, wherein the expression of the FvW protein was induced. Suitable examples thereof include all the animal or human cell lines transfected by means of a vector comprising the gene encoding this protein, preferably the gene encoding the human protein, where said cell lines may be especially selected from CHO-K, CHO-LeC10, CHO Lec-1, CHO Pro-5, CHO dhfr-, Wil-2, Jurkat, Vero, Molt-4, COS-7, 293-HEK, YB2/0, BHK, K61-16, NSO, SP2/0-Ag 14 and P3X63Ag8. 653, SK-Hep, HepG2 cell lines, as well as plant cells, bacteria systems, for example E. Coli, fungi systems, systems using viruses, especially baculoviruses, this list being non limitative.

Such cellular systems do express the FvW protein and may be obtained by means of methods well known from the man skilled in the art. U.S. Pat. No. 5,198,349 and WO 89/06096 may be mentioned as suitable examples, this list being non limitative. U.S. Pat. No. 5,198,349 describes human FvW expression in COS cells, by inserting the cDNA encoding human FvW into an expression vector.

When the solution is of transgenic origin, it is derived from a pluricellular system, especially from an animal or a plant obtained by transgenesis, that is to say wherein one or more cell(s) received a recombinant DNA molecule encoding FvW. Suitable examples thereof include dogs, cats, mice, rats, cows, goats, sheeps, rabbits and pigs, insects, plants, for example tobacco, soybean, this list being non limitative.

When FvW is produced by transgenic animals, this production may be effected in various media secreted by the animal, for example from urine, blood, saliva or milk, this list being non limitative. Such production methods may be carried out by means of methods well known from the man skilled in the art. WO 2001/022810 and WO1999/058699 are suitable examples thereof, this list being non limitative. In this respect, WO 2001/022810 describes the production of female transgenic mice which produce human FvW in their milk.

When FvW is produced by plants, this production may be effected by means of methods well known from the man skilled in the art, as described for example in U.S. Pat. No. 6,331,416 and U.S. Pat. No. 5,994,628, this list being non limitative.

When the solution is of plasmatic origin, it may be either an animal or a human plasma fraction, or a cryoprecipitate, or a fraction obtained by means of standard fractionation methods (Cohn and al., J. Am. Chem. Soc., 68, 459, 1946 and Kistler and al., Vox Sang., 7, 1962, 414-424). These fractions may optionally have been subjected to a pre-purification treatment such as the adsorption on aluminium hydroxide.

As used herein, a “solution derived from a secretion of a non-human animal containing FVIII” is intended to mean any solution derived from any secretion, for example from plasma, from saliva, from urine or from milk, produced by a transgenic animal that was engineered so as to express a FVIII molecule in one of the previously mentioned secretions. In this case, FvW is not expressed by the transgenic animal. As used herein, a “solution derived from FVIII-containing plasma” is intended to also include any solution derived from plasma naturally containing human or animal FVIII, that is substantially made free of FvW. It may be derived from an animal or a human plasma fraction, or a cryoprecipitate, or from a fraction obtained by standard fractionation methods (Cohn and al., J. Am. Chem. Soc., 68, 459, 1946 and Kistler and al., Vox Sang., 7, 1962, 414-424). These fractions may optionally have been submitted to a pre-purification treatment such as the adsorption on aluminium hydroxide.

Methods for producing secretions from a transgenic animal may be carried out by means well known from the man skilled in the art. Suitable examples thereof include U.S. Pat. No. 5,880,327 and US2007/0011752, this list being non limitative. In this respect, U.S. Pat. No. 5,880,327 describes the production of human FVIII in the milk of a transgenic mouse. US2007/0011752 describes the production of human proteins, for example FVIII, from the saliva of various animals.

As used herein, a “solution derived from a FVIII-containing plant extract” is intended to mean any fraction from a plant containing FVIII proteins, especially of human origin, obtained in a plant or a cell of a transgenic plant engineered so as to express a FVIII molecule.

Such an extract may be produced by introducing into a plant cell a nucleotide vector containing the gene encoding FVIII. Such methods are well known from the man skilled in the art, and may be illustrated by numerous documents that do belong to the state of the art, such as for example US 2005/0060775.

As used herein, an “ion-exchange chromatography filter-type membrane” is intended to mean any physical semipermeable barrier with the ability to adsorb FVIII and/or FvW by ion-exchange when these proteins are drawn through the membrane. In addition, this membrane can allow FVIII and FvW to flow through when the ion-exchange interaction between the membrane and FVIII and/or FvW is no more sufficient to retain them on the membrane.

Thus, to implement the method for purifying FVIII or FvW according to the invention, an ion-exchange filter-type membrane is used, made of a macroporous substrate comprising, fixed on said substrate, a negatively or positively charged coating, said coating providing the filter-type membrane with ion-exchange properties. Generally, the negatively or positively charged coating is fixed onto the macroporous substrate through chemical grafting.

The porosity of the macroporous substrate, that is to say the average pore size thereof, is such that the filter-type membrane does allow FVIII and FvW to flow through. A first benefit to use such a membrane relies on the possibility to use disposable exchange filter-type membranes, which improves the sanitary safety level of the method. A second benefit to use such a membrane relies on the possibility to conduct the purification method according to the invention, and more precisely the ion-exchange chromatography step(s), under a very high flow rate of the solution to be purified.

Any type of physical barrier substrate may be suitable for a filter-type membrane adapted to the implementation according to the invention, such as for example a polymer film, a wick, a hollow fiber, stabilized cellulose, polyethersulfone, or any three-dimensional structure that can allow FVIII and FvW to flow through.

The ion-exchange interaction between the filter-type membrane and the FVIII and FvW proteins results from the positively or negatively charged coating which is fixed onto the macroporous substrate, said coating comprising basic or acidic functional moieties that can be replaced.

The ion-exchange coating may be of the monofunctional type, that is to say comprising only one type of functional moiety, or of the polyfunctional type if there are various types of functional moieties.

Thus, the filter-type membrane enables to simultaneously capture or adsorb both FVIII and FvW proteins, thanks to the interaction between these proteins and the membrane. Upon applying a solution containing FVIII and FvW onto the membrane, FvW and FVIII are retained by the membrane thanks to ion-exchange interactions.

When the products to purify are not contained in a cryoprecipitate, it is preferred to perform a step of pre-purification of the solution to purify prior to carrying out the purification method according to the invention, so as to obtain a solution that has been sufficiently made free of contaminants.

This pre-purification step may be beneficial, especially with complicated solutions, for example derived from milk or from plasma, which is a medium containing a great number of proteins. Such a pre-purification step may especially comprise a clarification step, for example such as described in WO 2004/076695, or an extraction step such as described for example in FR 06 04864 or FR 06 11536, this list being non limitative. In this respect, FR 06 04864 describes an extraction method of at least one protein present in milk, said protein having an affinity for the calcium ions of said milk, whether complexed or not, comprising the following steps consisting in:
(i) releasing the protein by precipitating calcium compounds obtained by contacting the milk with a soluble salt, for example sodium phosphate, the anion of which is selected for its ability to form in a such medium said insoluble calcium compounds, so as to obtain a liquid phase enriched with the protein,
(ii) separating the liquid phase enriched with the protein from the calcium compound precipitate, said liquid phase being moreover separated to form a lipidic phase and an aqueous, non lipidic phase containing the protein, and
(iii) recovering the aqueous, non lipidic phase containing the protein.

Moreover, FR 06 11536 describes a method for extracting a protein present in milk, comprising at least one hydrophobic pocket and a negative charge at milk's natural pH, comprising the following steps of:
a) skimming and delipidating said milk,
b) applying the delipidated and skimmed fraction containing said protein onto a chromatographic substrate which is grafted with a ligand that has both a hydrophobic and an ionic character, for example 4-mercapto-ethyl-pyridine under pH conditions enabling said protein to be retained on said substrate,
c) eluting the protein,
d) purifying the eluted fraction by removing the milk proteins from said eluted fraction, and
e) recovering said protein.

In the case of solutions produced by cellular systems, the pre-purification step may be performed immediately following the cell culture step as such. The composition of the cell culture medium may be controlled so that the proteins produced by the cell are excreted in the extracellular medium. Moreover, the cell selection may be done so that the resulting protein is excreted in the medium.

Thereafter, a depth filtration step or a tangential micro-filtration may be needed for obtaining a pre-purification adapted to the implementation of the steps of the purification method according to the invention.

In some particular embodiments of the invention, the filter-type membrane is a cation-exchange selective membrane. Positive counter-ions of the functional groups carried by the membrane are replaced with charges of the same sign on FvW and FVIII.

Functional groups that may be used for the cation-exchange coating include carboxymethyl (CM), phosphoryl and sulfopropyl (SP), sulfate (S), this list being non limitative.

In these embodiments using a cation-exchange filter-type membrane, buffers that may be used include tris-hydroxymethyl-aminomethane, carbonate, ethylene diamine, imidazole or triethanolamine, this list being non limitative.

In these embodiments, the buffer pH value will be selected depending on the isoelectric point of the protein, so that the protein at such pH value has a positive global charge level, according to routine methods well known from the man skilled in the art.

In further embodiments according to the invention, the filter-type membrane implemented is an anion-exchange selective membrane. Negative counter-ions of the functional moieties carried by the membrane are replaced with charges of the same sign on FvW and FVIII.

Functional moieties that may be used for the anion-exchange coating include diethylaminoethyl (DEAE), diethyl (2-hydroxypropyl) aminoethyl or quaternary ammonium (QAE, Q), dimethylaminoethyl (DMAE) and trimethylaminoethyl (TMAE), this list being non limitative. In these embodiments, buffers that may be used are acetate, citrate, phosphate, glycine, barbiturate, this list being non limitative.

In these embodiments, the buffer pH value will be selected depending on the isoelectric point of the protein, so that the protein at such pH value has a negative global charge level, according to routine methods well known from the man skilled in the art.

Preferably, the filter-type membrane comprises an ion-exchange coating consisting in a strong anion exchanger.

As used herein, a “strong anion exchanger” is intended to mean any anion-exchange membrane capable of adsorbing poorly ionized proteins.

Such filter-type membranes are commercially available. Suitable examples include membranes carrying QAE or Q moieties, especially the Mustang Q® membrane (Pall) or the Sartobind Q membrane (Sartorius), this list being non limitative.

The Mustang Q membrane (Pall) is particularly interesting, since it does possess a high adsorption capacity and high chromatographic volume flows and since it can be used in a single cycle (disposable) or in multi-cycles.

Moreover, said membrane makes unnecessary to conduct the validation steps of substrate's washing, such as regeneration and sanitization (virus and prion security protocols, and the like).

Using such a filter-type membrane also makes unnecessary to perform aging studies that are traditionally conducted for standard substrates.

Thus, using such a membrane enables to reduce production times as compared to methods of the state of the art.

Preferably, the surface of the filter-type membrane that is contacting the solution to purify comprises an anion-exchange coating comprising quaternary ammonium groups that are fixed on the macroporous substrate by a chemical grafting.

In a further particular embodiment, the filter-type membrane is a macroporous membrane.

As used herein, the term “macroporous” is intended to mean a system composed of membrane pores which size does range from 0.3 μm to 1.0 μm. More preferably, the pore size does range from 0.5 μm to 0.9 μm. Most preferably, the pore size is 0.8 μm.

In a particularly preferred embodiment according to the invention, the substrate of the membrane is a polyethersulfone substrate.

As an example, such a polyethersulfone membrane may be a Mustang Q® membrane, marketed by Pall. This membrane has 0.8 μm-diameter pores and it is grafted with quaternary amine groups.

In a particular embodiment according to the invention, the solution to purify contains FVIII or FvW, and is of plasmatic origin.

In a preferred embodiment according to the invention, the solution to purify contains a mixture of FVIII and FvW, and is more preferably derived from plasma.

Thus, in these embodiments according to the invention, the solution is derived from natural, human or animal plasma, that is to say from a human or animal plasma that does naturally contain human or animal FVIII and FvW, respectively. Such an animal plasma may be collected in pigs, rabbits, goats, this list being non limitative.

Surprisingly, the applicant observed that filter-type membranes, and especially the Mustang Q® membranes do have a FVIII and/or FvW adsorption capacity higher than that of a resin or a gel. As used herein, the “adsorption capacity” is intended to mean the amount of target proteins fixed on the gel, and it is generally expressed by the gel or resin manufacturer in BSA amount (bovine serum albumin) fixed on the gel for anionic resins. For example, it does range from 25 to 35 mg/ml for the DEAE-TOYOPEARL® gel, traditionally used for purifying FVIII and/or FvW, while it is higher than or equal to 30 mg/ml for the Mustang Q® membrane.

This higher adsorption capacity of the filter-type membrane means the possibility to use a smaller amount of gel equivalent to adsorb a same amount of FVIII and/or FvW as compared to a gel or a resin. As an illustration, it is possible to adsorb from 50 to 80 IU FvW and FVIII/ml of gel on DEAE and from 200 to 250 IU VWF and FVIII/ml of gel equivalent on a Mustang Q® membrane.

The highest adsorption capacity of the filter-type membrane as compared to a gel or a resin could result from an improved accessibility for FVIII and/or FvW to the ionized sites, especially when they form FVIII/FvW complexes. Indeed, these proteins have a large size, especially when they form FVIII/FvW complexes, and we can assume that the ionized sites are more easily accessible to them when they are grafted onto the macroporous filter-type membrane, due to the pore size, than onto gels or resins, which ionized sites are embedded into channels, making them less accessible to large proteins.

Advantageously, in this preferred embodiment according to the invention, the method comprises following steps of:
a) providing a cryoprecipitate from plasma,
b) capturing, that is to say adsorbing, factor VIII and Von Willebrand factor on said ion-exchange chromatography membrane, and more particularly on the anion-exchange chromatography membrane, and
c) selectively recovering Von Willebrand factor and factor VIII by successively increasing the ionic strength value of the elution buffer.

Advantageously, after the step of providing a cryoprecipitate from plasma, it can be performed a pre-purification step by adsorption of factor VIII and Von Willebrand factor on an alumina gel and then a cold precipitation.

In step c) of the hereabove method, the ionic strength value of the elution buffer is easily adjusted by the one skilled in the art, based on his general knowledge about the physico-chemical properties of each of the FvW and FVIII proteins and about ion-exchange properties of the filter-type membrane, which is generally a commercially available filter-type membrane, for which use recommendations are provided by the manufacturer. The one skilled in the art especially knows, based on his general knowledge, that FVIII is eluted from an anion-exchange chromatography substrate with a buffer having an ionic strength value higher than the ionic strength value necessary for eluting FvW from the same substrate.

Thus, in step c) of the hereabove method, the one skilled in the art does use a buffer with an ionic strength value suitable for eluting (desorbing from the substrate) (i) FvW, (ii) FvW and FVIII or (iii) successively first FvW, then FVIII. According to a first embodiment of the alternative (iii) consisting in successively eluting firstly FvW, then FVIII, the one skilled in the art may successively use two elution buffers, each elution buffer having an ionic strength value adjusted to the desorption of FvW or FVIII, respectively. According to a second embodiment of the alternative (iii), the one skilled in the art does perform the elution with a buffer capable of generating an increasing ionic strength gradient with which FvW, then FVIII are successively desorbed.

Thus, as used herein, “successive increases in the ionic strength value of the elution buffer”, for step c) of the hereabove method does include a linear increase in the ionic strength value of the elution buffer, that may be obtained by adding a salt, for example sodium chloride, calcium chloride, this list being non limitative.

One can obtain first the FvW elution and then FVIII elution by increasing the ionic strength value of the buffer. It is therefore thus possible either to recover FvW and to stop the elution after having obtained FvW, either to obtain FVIII by continuing the elution.

As used herein, a “cryoprecipitate” is intended to mean a precipitate obtained from a human or an animal plasma by means of a low temperature-precipitation method. The cryoprecipitate may be obtained using methods well known from the man skilled in the art. As an example, frozen plasma is brought to a temperature of about −5° C. to −15° C., then slowly warmed up under stirring to a temperature not exceeding 1° C. or optionally 4° C. Under these conditions, frozen plasma melts down to give a liquid phase and a solid phase. The solid phase, i.e. the cryoprecipitate, is then recovered by centrifugation. The cryoprecipitate is substantially composed of fibrinogen, fibronectin, factor VIII and Von Willebrand factor (vWF). In the cryoprecipitate, FVIII is generally associated with FvW which stabilizes FVIII.

As used herein, a “selective recovery” is intended to mean the ability to recover either FVIII, or FvW, or a mixture of FVIII and FvW, depending on the elution method, depending on the expected protein(s).

It is possible to obtain either FvW, or FVIII, or a mixture of FVIII and FvW, by changing the pH value, or by increasing the ionic strength value of the elution buffer according to a method well known from the one skilled in the art (see for instance example 1).

In a further embodiment, the solution to purify contains a mixture of FVIII and FvW of recombinant or transgenic origin.

This embodiment comprises the following steps of:
a) providing a cell culture supernatant or a prepurified solution comprising FVIII and FvW,
b) capturing factor VIII and Von Willebrand factor on said ion-exchange chromatography membrane, and more particularly on the anion-exchange chromatography membrane, and
c) selectively recovering Von Willebrand factor and factor VIII by successively increasing the ionic strength value of the elution buffer.

In a further embodiment according to the invention, the solution to purify contains either FVIII, or FvW.

This embodiment comprises the following steps of:
a) providing a cell culture supernatant or a purified solution containing FVIII or FvW,
b) capturing factor VIII or Von Willebrand factor on said ion-exchange chromatography membrane, and more particularly on the anion-exchange chromatography membrane, and
c) Recovering Von Willebrand factor or factor VIII.

Advantageously, with a plasma fraction, the method of the invention enables to reduce the amount of vitamin-K dependent factors, of fibrinogen and of fibronectin.

Advantageously, especially when the solution to purify contains a mixture of FVIII and FvW, the selectively recovery of FvW and FVIII is effected by means of the following steps of:

c1) eluting FvW by increasing the ionic strength value of the equilibration buffer of said chromatography membrane. For example, the ionic strength value may be increased by adding sodium chloride 0.25 M to reach an osmolality ranging from 600 to 660 mOsm/Kg approximately.

c2) eluting FVIII by increasing the ionic strength value of the equilibration buffer of said chromatography membrane even more than for recovering Von Willebrand factor. For example, the ionic strength value may be increased by adding sodium chloride 0.7 M, or calcium chloride 0.35 M, to reach an osmolality ranging from 1400 to 1700 mOsm/Kg approximately.

Step c1) is a step of FvW selective recovery, during which a buffer solution is used, that has an ionic strength value suitable for desorbing FvW from the ion-exchange filter-type membrane.

Step c2) is a step of FVIII selective recovery, during which a buffer solution is used, that has an ionic strength value suitable for desorbing FVIII from the ion-exchange filter-type membrane. In step c2), a buffer solution is used that has an ionic strength value higher than the ionic strength value of the buffer solution used for step c1).

These steps are performed after FVIII and FvW simultaneous capture step on the ion-exchange chromatography filter-type membrane.

FvW eluted in step c1) contains very few FVIII. It is recovered and a purified FvW solution is obtained.

Moreover, FVIII obtained in step c2) contains less FvW than the initial solution. It is recovered and a FVIII purified solution is obtained.

Optionally, it is possible to implement an additional step so as to dissociate high molecular weight FVIII-FvW complexes, for example such as described in EP 1 037 923. Optionally, it is then possible to implement a step of additional filtration for the FVIII purified solution, on a hydrophilic filter with a porosity lower or equal to 20 nm, especially equal to 15 nm, as described in WO 2005/040214.

In a further particular embodiment, the method of the invention enables to obtain a purified FvW solution, by performing, after FVIII and FvW simultaneous adsorption step on said ion-exchange chromatography filter-type membrane, the following steps of:
d) eluting FvW by increasing the ionic strength value of the equilibration buffer of said chromatography membrane,
e) capturing Von Willebrand factor on an ion-exchange chromatography membrane, more preferably of the same type as the first membrane, and
f) eluting Von Willebrand factor by increasing the ionic strength value of the equilibration buffer of said chromatography membrane.

In this embodiment, it is optionally possible, after FvW elution step d) by increasing the ionic strength value of the equilibration buffer of said chromatography membrane, to elute FVIII by increasing the ionic strength value of the equilibration buffer of said chromatography membrane even more than for the recovery of Von Willebrand factor. Thus, in a simplified method, both a purified FvW-containing solution and a purified FVIII-containing solution are obtained.

The method of the invention thus advantageously enables to obtain the sequential or simultaneous purification of FVIII and FvW from a solution containing FVIII and FvW. If the solution is of plasmatic origin, the method according to the invention is particularly advantageous because it enables to take beneficial the use of human or animal plasma as much as possible.

Advantageously, this particular embodiment comprises in addition the following steps of:
g) performing the chromatography of the Von Willebrand factor-enriched fraction which was eluted in step c) on an affinity gel column with a gelatin ligand, and
h) recovering the Von Willebrand factor fraction that is non retained and devoid of fibronectin.

The fibronectin assay may be performed for example by immuno-nephelometry, according to a method well known from the one skilled in the art.

Thus, an embodiment of the method according to the invention enables to obtain a purified FVIII solution and a purified FvW solution, by performing, after FVIII and FvW simultaneous capture step on the ion-exchange chromatography filter-type membrane, the following steps of:
a) eluting FvW by increasing the ionic strength value of the equilibration buffer of said chromatography membrane,
b) eluting FVIII by increasing the ionic strength value of the equilibration buffer of said chromatography membrane even more than for the recovery of Von Willebrand factor,
c) capturing Von Willebrand factor on an ion-exchange chromatography membrane,
d) eluting Von Willebrand factor by increasing the ionic strength value of the equilibration buffer of said chromatography membrane,
e) performing the chromatography of the Von Willebrand factor-enriched fraction eluted in step d) on an affinity gel column with a gelatin ligand, and
f) recovering the Von Willebrand-enriched fraction that was not retained on le gel affinity.

Thus, in this embodiment, a purified FVIII solution and a purified FvW solution are successively obtained.

The method according to the invention, in its various embodiments, enables thus to separate FVIII and FvW in a simplified manner.

The purification steps according to the invention are the only ones which enable to separately or simultaneously purify factor VIII and Von Willebrand factor from plasma, by simultaneously capturing both proteins on an anion-exchange chromatography selective membrane.

It is a further object of the present invention to provide a method for preparing purified FVIII comprising the implementation of the purification method according to the invention.

It is a further object of the present invention to provide a method for preparing purified FvW comprising the implementation of the purification method according to the invention.

Further aspects and advantages of the invention will be described in the following examples which are meant for illustrative purposes only and should not in any way limit the scope of the invention.

### FIGURES

FIG. 1: diagram of the Von Willebrand factor purification method.

FIG. 2: diagram of the factor VIII purification method.

### EXAMPLES

### Example 1

### Purification Method of Von Willebrand Factor

A cryoprecipitate is prepared by thawing fresh frozen plasma to a temperature lying between 1° C. and 6° C.

After centrifugation, the cryoprecipitate containing fibrinogen, fibronectin, Von Willebrand factor and factor VIII is recovered and slurried in an aqueous solution containing sodium heparin (3 IU/mL). The pH value of the solution is then adjusted to 7.0±0.1.

The slurried cryoprecipitate is subjected to a pre-purification by adsorption on alumina gel to remove vitamin-K dependent factors and by fibrinogen and fibronectin cold precipitation. Thus, aluminium hydroxide is added to the suspension under stirring for 5 minutes. The pH value is adjusted to 6.5±0.2 with acetic acid 0.1M and the solution is cooled down under stirring until the temperature does range from 14 to 18° C. The solution is then centrifuged to a temperature of 14-18° C. The supernatant is recovered and clarified by filtration on a 0.22 μm filter.

This prepurified solution is then subjected to a virus inactivation step by a treatment with solvent/detergent in the presence of Polysorbate 80 (1%, w/v) and Tri-n-Butyl Phosphate (0.3%, v/v) efficient against enveloped viruses. The treatment with solvent/detergent is performed for a time period of at least 6 hours at a pH value of 7.1.

The solvent/detergent-treated protein solution does then pass through a strong anion-exchange grafted membrane, such as the Mustang Q capsule, previously equilibrated with a basic buffer solution, pH 6.9-7.1 that was enriched with sodium chloride to reach an osmolality ranging from 370 to 390 mOsm/Kg.

Once the protein solution has gone through, the capsule is rinsed with the same buffer solution (osmolality 370-390 mOsm/Kg) until the optical density of the column effluent is back to baseline. The protein fraction that was not adsorbed on the membrane contains much fibrinogen as well as the chemical agents added to for the solvent/detergent-based virus inactivation treatment.

Von Willebrand factor adsorbed on the membrane is then eluted by applying the basic buffer solution, pH 6.9-7.1 with an ionic strength value increased by adding sodium chloride to reach an osmolality of 600-660 mOsm/Kg. The eluted fraction is then diluted with the basic buffer solution, pH 6.9-7.1 devoid of sodium chloride until an osmolality ranging from 370 to 390 mOsm/Kg is reached.

The diluted fraction does then pass through a strong anion-exchange grafted membrane, of the Mustang Q capsule type, previously equilibrated with the basic buffer solution, pH 6.9-7.1 that was enriched with sodium chloride to reach an osmolality ranging from 370 to 390 mOsm/Kg. Once the protein solution has gone through, the capsule is rinsed using the same buffer solution (osmolality 370-390 mOsm/Kg) until the optical density of the column effluent is back to baseline. Von Willebrand factor adsorbed on the membrane is then eluted by applying the basic buffer solution, pH 6.9-7.1 with an ionic strength value increased by adding sodium chloride to reach an osmolality of 600-660 mOsm/Kg. The eluted fraction is then submitted to an affinity chromatography on gel using a gelatin ligand previously equilibrated with a basic buffer solution, pH 6.9-7.1 with an ionic strength value increased by adding sodium chloride to reach an osmolality of 600-660 mOsm/Kg. Once the protein solution has gone through, the affinity gel is rinsed using the same buffer solution (osmolality 600-660 mOsm/Kg) until the optical density of the column effluent is back to baseline. The non adsorbed fraction including the gel washing represents the highly pure Von Willebrand factor-enriched fraction.

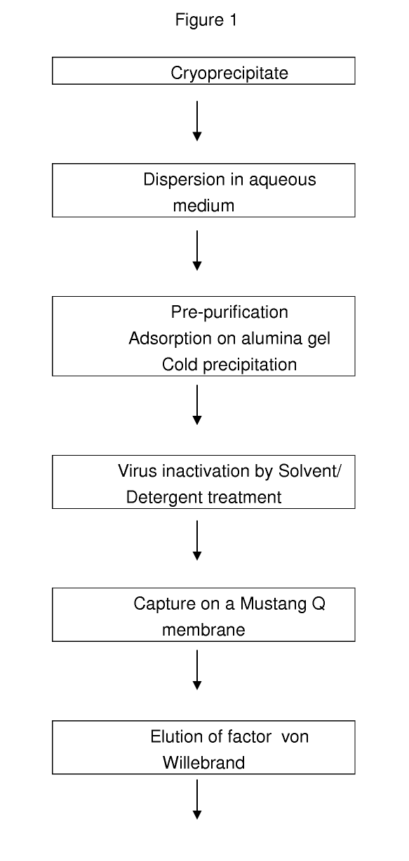

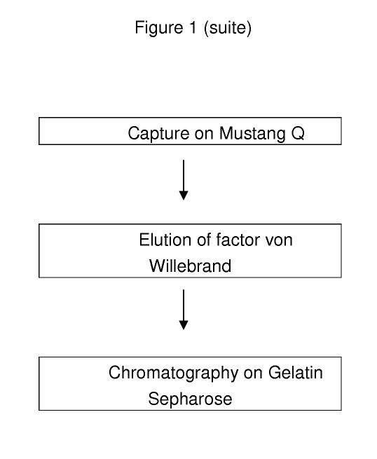

The purification diagram is illustrated on FIG. 1.

### Results

TABLE 1
Purification of Von Willebrand factor

| | Yield | Specific activity |
|---|---|---|
| Step | (%) | (IU/mg) |
| Initial cryoprecipitate | 100 | 0.46 |
| 1^{st} purification step on | ≧30 (30-50) | 20-30 |
| Mustang Q XT5 capsule | | |
| 2^{nd} purification step on | ≧70 | >80 |
| Mustang Q XT5 capsule + | | |
| chromatography on gelatin- | | |
| Sepharose | | |

### Example 2

### Method for Purifying Factor VIII

A cryoprecipitate is prepared by thawing fresh frozen plasma to a temperature lying between 1° C. and 6° C.

After centrifugation, the cryoprecipitate containing fibrinogen, fibronectin, Von Willebrand factor and factor VIII is recovered and slurried in an aqueous solution containing sodium heparin (3 IU/mL). The pH value of the solution is then adjusted to 7.0±0.1.

The slurried cryoprecipitate is subjected to a prepurification by adsorption on alumina gel to remove vitamin-K dependent factors and by fibrinogen and fibronectin cold precipitation. Thus, aluminium hydroxide is added to the suspension under stirring for 5 minutes. The pH value is adjusted to 6.5±0.2 with acetic acid 0.1M and the solution is cooled down under stirring until the temperature does range from 14 to 18° C. The solution is then centrifuged to a temperature of 14-18° C. The supernatant is recovered and clarified by filtration on a 0.22 μm filter.

This prepurified solution is then submitted to a virus inactivation step by a treatment with solvent/detergent in the presence of Polysorbate 80 (1%, w/v) and Tri-n-Butyl Phosphate (0.3%, v/v) efficient against enveloped viruses. The treatment with solvent/detergent is conducted for a time period of at least 6 hours at a pH value of 7.1.

The solvent/detergent-treated protein solution does then pass through a strong anion-exchange grafted membrane, of the Mustang Q capsule, previously equilibrated with a basic buffer solution, pH 6.9-7.1 that was enriched with sodium chloride to reach an osmolality ranging from 370 to 390 mOsm/Kg.

Once the protein solution has gone through, the capsule is rinsed using the same buffer solution (osmolality 370-390 mOsm/Kg) until the optical density of the column effluent is back to baseline. The protein fraction that was not adsorbed on the membrane contains much fibrinogen as well as the chemical agents added to for the solvent/detergent-based virus inactivation treatment.

Von Willebrand factor adsorbed on the membrane is then eluted by applying the basic buffer solution, pH 6.9-7.1 with an ionic strength value increased by adding sodium chloride to reach an osmolality of 600-660 mOsm/Kg. The purification of Von Willebrand factor may be continued as described in example 1. Factor VIII adsorbed on the membrane is then eluted by applying the basic buffer solution, pH 6.9-7.1 with an ionic strength value increased by adding sodium chloride to reach an osmolality de 1400-1700 mOsm/Kg. Advantageously, factor VIII is eluted with a buffer solution, pH 6.0, having a high ionic strength value obtained by adding calcium chloride.

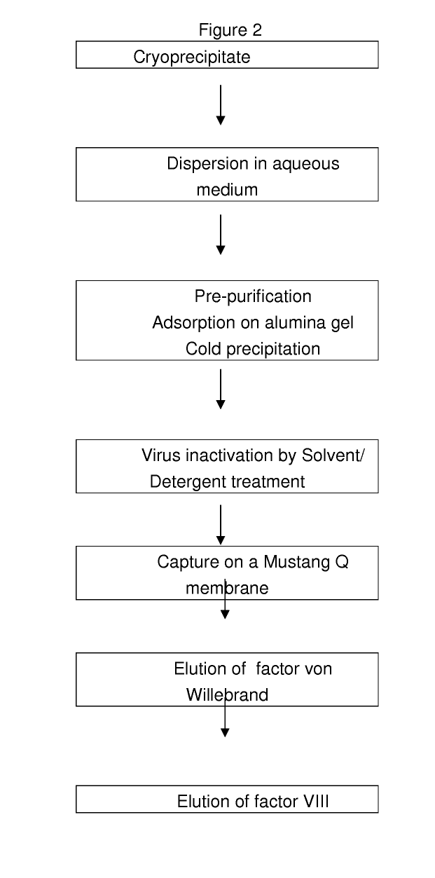

The purification diagram is illustrated on FIG. 2.

### Results

TABLE 2
Purification of factor VIII

| | Yield | Specific activity |
|---|---|---|
| Step | (%) | (IU/mg) |
| Initial cryoprecipitate | 100 | 0.38 |
| Purification step on | ≧45 (40-60) | ≧110 |
| Mustang Q XT5 capsule | | |

1. A method for purifying FVIII or FvW from a solution selected from (i) a solution containing a mixture of FVIII and FvW, (ii) a solution containing FvW, (iii) a solution derived from a secretion of a non-human animal and (iv) a solution derived from a FVIII-containing plant extract, said method being characterized in that it comprises a step of adsorption of FVIII or FvW on an ion-exchange chromatography filter-type membrane.

2. A method according to claim 1, wherein said filter-type membrane is an anion-exchange chromatography membrane.

3. A method according to claim 1, wherein said filter-type membrane is a strong anion exchanger.

4. A method according to claim 1, wherein said membrane is provided with a coating comprising quaternary amine groups.

5. A method according to claim 1, wherein said membrane is a macroporous-type membrane.

6. A method according to claim 1, wherein said membrane is a polyethersulfone-type membrane.

7. A method according to claim 1, wherein said solution contains a mixture of FVIII and FvW.

8. A method according to claim 7, wherein said solution is of plasmatic origin.

9. A method according to claim 8, which comprises the following steps of:
a) providing a cryoprecipitate from plasma,
b) adsorbing factor VIII and Von Willebrand factor on said ion-exchange chromatography membrane,
c) recovering factor VIII or Von Willebrand factor by using an elution buffer having a suitable ionic strength value.

10. A method according to claim 9, wherein said selective recovery of Von Willebrand factor and factor VIII is performed according to the following steps of:
c1) selectively recovering FvW by elution with a buffer having a suitable ionic strength value,
c2) selectively recovering FVIII by elution with a suitable buffer having an ionic strength value higher than the ionic strength value of the buffer used in step c1).

11. A method according to claim 1, which comprises the additional following steps:
d) eluting FvW by increasing the ionic strength value of the equilibration buffer of said chromatography membrane,
e) capturing Von Willebrand factor on an ion-exchange chromatography membrane, more preferably of the same type as the first membrane,
f) eluting Von Willebrand factor by increasing the ionic strength value of the equilibration buffer of said chromatography membrane.

12. A method according to claim 11, which further comprises the following steps of:
g) performing the chromatography of the Von Willebrand factor-enriched fraction eluted in step c) on an affinity gel column with a gelatin ligand,
h) recovering the non retained Von Willebrand factor fraction that is devoid of fibronectin.

13. A method for producing purified FVIII, which comprises the implementation of the method according to claim 1.

14. A method for producing purified FvW, which comprises the implementation of the method according to claim 1.
